(19) United States
(12) Patent Application Publication
(10) Pub. No.: US 2006/0252069 A1
(43) Pub. Date: Nov. 9, 2006

(54) PCR FOR MRSA SCCMEC TYPING

(76) Inventors: Kunyan ZHANG, Calgary, AB (CA); John CONLY, Calgary, AB (CA)

Correspondence Address: EDWARD YOO C/O BENNETT JONES, 1000 ATCO CENTRE, 10035 - 105 STREET, EDMONTON, ALBERTA, AB, T5J3T2, CA

(21) Appl. No.: 11/379,676

(22) Filed: Apr. 21, 2006

Related U.S. Application Data
(60) Provisional application No. 60/673,358, filed on Apr. 21, 2005.

(51) Int. Cl. C12Q 1/68; C12P 19/34

(52) U.S. Cl. 435/6; 435/91.2

(57) ABSTRACT

A multiplex PCR assay for the detection, identification and classification of SCCmec types and sub-types of Staphylococcal aureus has been described.

### FIELD OF THE INVENTION

The present invention relates to a multiplex polymerase chain reaction (PCR) assay for Staphylococcus aureus typing. In particular, the invention relates to identification, detection and classification of all currently described staphylococcal cassette chromosome mec (SCCmec) types and subtypes.

### BACKGROUND

Methicillin-resistant Staphylococcus aureus (MRSA) strains were soon identified (Barber, 1961; Jevons, 1961) after the introduction of methicillin, which itself was developed to overcome resistance to penicillin. MRSA strains have acquired and integrated into their genome a 21 kb to 67 kb mobile genetic element, termed the staphylococcal cassette chromosome mec (SCCmec), which harbours the methicillin resistance (mecA) gene and other antibiotic resistance determinants (Ito, 2001 and 2004; Ma, 2002). MRSA strains have spread and become established as major nosocomial pathogens worldwide (Ayliffe, 1997; Crossley, 1979; Fluit, 2001; Panlilio, 1992; Voss, 1994). These organisms have evolved and emerged as a major cause of community-acquired infections (Lindsay, 2004; Vandenesch, 2003). These newly emerging community-acquired (C)-MRSA strains possess a novel small mobile SCCmec types IV or V genetic element which contains the mecA gene with or without other additional antibiotic resistance genes and is more easily transferred to other strains of S. aureus compared with larger SCCmec (types I, II and III) elements (O'Brien, 2004; Vandenesch, 2003). The emerging spread of these C-MRSA strains poses a significant threat to public health (Lindsay, 2004; Vandenesch, 2003).

A thorough understanding of the molecular epidemiology and evolution of MRSA is required to help detect, track, control and prevent human disease due to this organism. Full characterization of MRSA requires definition of not only the putative bacterial genetic background but also of the complex and heterologous SCCmec elements. SCCmec typing is one of the most important molecular tools available for understanding the epidemiology and clonal strain relatedness of MRSA, particularly with the emerging outbreaks of community-acquired MRSA occurring on a worldwide basis (Lindsay, 2004; O'Brien, 2004; Vandenesch, 2003). However, due to the very complex and diverse structure of the SCCmec element, SCCmec typing is usually achieved by DNA sequence analysis (21-67 kb) (Ito, 2001; Ito, 1999; Oliveira, 2001), Southern blot analysis using three or more restriction enzymes and several key probes specific for each SCCmec type (Oliveira, 2001), and by PCR.

Oliveira and de Lencastre developed a multiplex PCR strategy (Oliveira, 2002) for mec element type assignment and defined types of SCCmec based on genes located within the J-regions of SCCmec elements as follows: locus A, located downstream of the pls gene and is specific for SCCmec type-1; locus B, internal to the kdp operon, which is specific for SCCmec type II; locus C, internal to the mecI gene present in SCCmec types II and III; locus D, internal to the dcs region present in type-I, II, and IV; locus E, located in the region between integrated plasmid pI258 and transposon Tn554, specific for SCCmec type III; locus F, which is also specific for SCCmec type-III located in the region between Tn554 and orf X; locus G, the left junction between IS431 and pUB110; and locus H, the left junction between IS431 and pT181 (Oliveira, 2002). This is the only single step multiplex PCR assay known to the inventors but it too has its limitations. However, being simpler and easier to perform than the traditional (non-multiplex) PCR assays for SCCmec typing, it has been increasingly used in favor of the traditional method. As a result, different SCCmec types are named according to the standard SCCmec type definition first established by Hiramatsu's group (Ito et al, 2004). In addition to hampered interpretation due to the presence of multiple bands for each SCCmec type (because of non-type-specific targets) and difficulties in assay optimization, Oliveira's assay has limitations in detecting the newly described SCCmec type V, mis-classifying them as type III (Table 3), while failing to discriminate type IV into subtypes IVa, b, c and d (Oliveira, 2002). Since the newer SCCmec types IV and V have recently been associated with community-aquired infection (Ito, 2004; Vandenesch, 2003), detecting type V, and discriminating type IV into subtypes IVa, b, c and d will play an important role in the prevention and control of currently emerging community MRSA clonal outbreaks. Therefore, a more robust and simpler SCCmec typing assay is required.

The new MRSA nomenclature scheme recently set by the International Union of Microbiology Societies incorporates SCCmec typing information in conjunction with that provided by multilocus sequence typing (MLST) (Enright, 2002; Robinson, 2003 and 2004).

Previously described traditional PCR SCCmec typing schemes target the individual regions of the classes of the mec-complex (IS431-mecA, IS1272-mecA, mecI-mecRI), the allotypes of the ccr-complex (ccrA1, ccrA2, ccrA3, ccrB1, ccrB2, ccrB3 and ccrC), and individual subtypes of the J regions, and therefore require the use of many (20 to 30) primer sets and multiple individual PCR experiments (Ito 2004; Okuma, 2002). The only previously described multiplex PCR assay for SCCmec typing (Oliveira, 2002) is more difficult to interpret and is limited in its ability to detect SCCmec subtypes IVa, b, c and d plus the newly described type V, these groups being implicated in currently emerging community MRSA outbreaks (Ito, 2004, Vandenesch, 2003). These methods are laborious, time-consuming and expensive, resulting in limited utility for clinical and surveillance purposes.

Hence, there is a need in the art for development of a multiplex PCR assay capable of detecting and classifying all currently described SCCmec types and subtypes, with simultaneous discrimination of MRSA from methicillin-susceptible S. aureus (MSSA).

### SUMMARY OF THE INVENTION

The present invention relates to a multiplex PCR assay for staphylococcal species. In a preferred embodiment, an assay for the detection, identification and classification of SCCmec types and sub-types. In one embodiment, the invention comprises oligonucleotides sequences that may be used as primers for the detection, identification and classification of SCCmec types and sub-types.

Therefore, in one aspect, the invention may comprise a multiplex PCR SCCmec typing assay for Staphylococcus aureus SCCmec types I, II, III, subtypes IVa, IVb, IVc, IVd and V, and MRSA and MSSA, comprising the steps of:
(a) obtaining an isolate of a sample of S. aureus;
(b) amplifying a loci unique to each type and subtype by a PCR technique using amplification primers selective for each of said loci; and
(c) detecting the PCR amplicons, and determining the type and subtype of the S. aureus isolate.

In another aspect, the invention may comprise an assay kit, comprising amplification primers described herein.

### BRIEF DESCRIPTION OF THE DRAWINGS

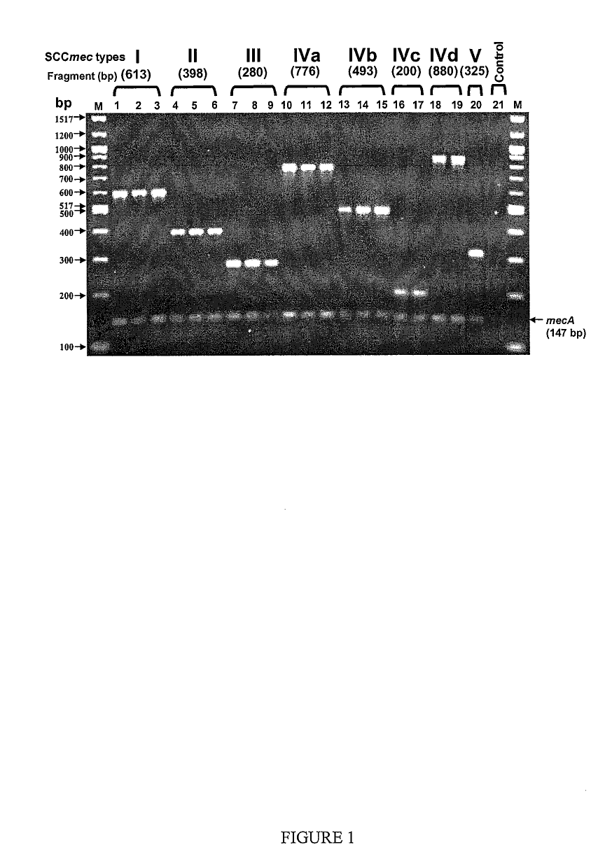

Exemplary embodiments of the invention may be described with reference to FIGURE 1 which shows the results of a multiplex PCR assay of the present invention, which identifies SCCmec types and subtypes I, II, III, IVa, IVb, IVc, IVd and V, and simultaneously detects the methicillin resistance (mecA gene). Type I, lanes 1-3 (strains NCTC10442, COL and PER34); type II, lanes 4-6 (strains N315, CLS-5153 and CLS-440); type III, lanes 7-9 (strains 85/2082, ANS46 and CMRSA-3); type IVa, lanes 10-12 (strains CA05, N02-590 and CLS-2207); type IVb, lanes 13-15 (strains 8/6-3P, CLS-4584 and CLS-5827); type IVc, lanes 16-17 (strains MR108 and CLS-1040); type IVd, lanes 18-19 (strains JCSC4469 and CMRSA-5); type V, lane 20 (strain WIS [WBG8318]-JCSC3624); lane 21, PCR negative control; and lanes M, molecular weight marker, 100-bp DNA Ladder (BioLabs), respectively. Refer to Table 3 for details of each strain.

### DETAILED DESCRIPTION OF PREFERRED EMBODIMENTS

When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

As used herein, “polymerase chain reaction” or “PCR” is a molecular biology technique for enzymatically replicating DNA without using a living organism, such as E. coli or yeast. The technique allows for small amount of the DNA molecule to be amplified many times, in an exponential manner, with more DNA templates available after every cycle.

As used herein, a “multiplex polymerase chain reaction” or “multiplex PCR” is a PCR reaction where more than one primer set is included in the reaction pool allowing 2 or more different targets to be amplified by PCR in a single reaction tube.

As used herein, a “primer” is an oligonucleotide or pair of oligonucleotides used to direct an activity to a region of nucleic acid. With PCR, a primer or pair of primers define the area of the genome to be amplified.

The present invention comprises new sets of SCCmec type- and subtype-unique and specific primers and at least one new set of methicillin resistance (mecA gene-based) primers. The novel primers of the present invention were developed with comprehensive analyses and alignments of the MSSA and MRSA genomes and SCCmec sequences. The primers are used in the novel multiplex PCR SCCmec typing assays of the present invention (in a single multiplex PCR reaction with a single band for each type or subtype), capable of classifying MRSA isolates into SCCmec types and subtypes I, II, III, IVa, IVb, IVc, IVd and V, according to the current updated SCCmec typing system, while simultaneously being able to discriminate MRSA from MSSA; as illustrated in the Examples herein.

It has only recently been shown that some methicillin-susceptible staphylococci, including MSSA and methicillin-susceptible coagulase-negative staphylococci, could harbour SCC elements that contain the essential features of SCCmec but lack the mecA gene (Corkill, 2004; Katayama, 2003; Luong, 2002; Mongkolrattanothai, 2004). These SCC elements serve as a vehicle of transfer for various genetic markers including genes mediating antibiotic resistance or virulence. The potential role of SCC for mediating gene movement in staphylococci is awaiting further investigation. Hence, the multiplex assay of the present invention (incorporating a concomitant mecA gene into specific SCCmec typing system) may play a critical role in this regard.

The assays of the present invention were designed to target the SCCmec type- and subtype-unique and specific gene loci, based on the currently available sequence data of the MRSA and MSSA genomes and variable SCCmec type and subtype sequences in the GenBank database.

SCCmec is a mobile genetic element characterized by the presence of terminal inverted and direct repeats, two essential genetic components (the mec gene complex and the ccr gene complex), and the junkyard (J) regions (Ito, 2001 and 2004; Ma, 2002). The mec gene complex is composed of IS431mec, mecA, and intact or truncated sets of regulatory genes, namely mecR1 and mec1. The ccr gene complex encodes the recombinases (ccr) that mediate the integration of SCCmec into and its excision from the recipient chromosome and are, therefore, responsible for its mobility. The rest of the SCCmec element is comprised of J regions (J1, J2, J3) that are located between and around the mec- and ccr-complexes and contain various genes or pseudo genes the presence of which does not appear to be essential or useful for the bacterial cell, although notable exceptions include plasmid- or transposon-mediated resistance genes for non-β-lactam antibiotics or heavy metals (Ito, 2003). So far, there are 3 classes (A, B and C) of mec-complex and 4 allotypes (type 1, 2, 3 and 5) of ccr-complex. Different combinations of these complex classes and allotypes generate various SCCmec types (Table 1). SCCmec elements are currently classified into types I, II, II, IV and V based on the nature of the mec- and ccr-gene complexes, and are further classified into subtypes according to differences in their J region DNA (Ito, 2001 and 2004; Ma, 2002).

TABLE 1
Current SCCmec types and type IV subtypes

| SCCmec | | | | |
|---|---|---|---|---|
| types and | | | | |
| subtypes^{a} | mec-complex^{b} | ccr-complex^{c} | Original Strains | GenBank No. & Reference |
| I | Class B mec | Type 1 ccr | NCTC10442 | AB033763 (Ito, 2001) |
| II | Class A mec | Type 2 ccr | N315 | D86934 (Ito, 2001) |
| III | Class A mec | Type 3 ccr | 85/2082 | AB37671 (Ito, 2001) |
| IVa | Class B mec | Type 2 ccr | CA05 | AB063172 (Ma, 2002) |
| IVb | Class B mec | Type 2 ccr | 8/6-3P | AB063173 (Ma, 2002) |
| IVc | Class B mec | Type 2 ccr | MR108 | AB096217 (Ito, 2003) |
| IVd | Class B mec | Type 2 ccr | JCSC4469 | AB097677 |
| V | Class C mec | Type 5 ccr | WIS | AB12121 (Ito, 2004) |
| | | | [WBG8318]- | |
| | | | JCSC3624 | |

^{a}Subtypes of SCCmec IV differ based on the Junkyard (J) region DNA.
^{b}Class A mec: IS431-mecA-mecR1-mecI; Class B mec: IS431-mecA-ΔmecR1-IS1272; Class C mec: IS431-mecA-ΔmecR1-IS431.
^{c}Type 1 ccr: ccrB1-ccrA1; Type 2 ccr: ccrB2-ccrA2; Type 3 ccr: ccrB3-ccrA3; Type 5 ccr: ccrC.

New sets of SCCmec type- and subtype-unique and specific primers, as well as the novel specific primers for mecA gene, and for typing mec- and ccr-gene complexes were designed based on the comprehensive analyses and alignments of the S. aureus and MRSA genomes and SCCmec sequences currently available in the GenBank database (National Center for Biotechnology Information, USA; updated as of December, 2004).

TABLE 2

Primers

| | | | | | |
|---|---|---|---|---|---|
| | | | Concent | Amplicon | |
| | | SEQ ID | ration | size | |
| Primer | Oligonucleotide Sequence (5′-3′) | NO | (uM) | (bp) | Specificity |

| | | | | | |
|---|---|---|---|---|---|
| Type I-F | GCTTTAAAGAGTGTCGTTACAGG | 1 | 0.048 | 613 | SCCmec I |
| Type I-R | GTTCTCTCATAGTATGACGTCC | 2 | | | |
| Type II-F | CGTTGAAGATGATGAAGCG | 3 | 0.032 | 398 | SCCmec II |
| Type II-R | CGAAATCAATGGTTAATGGACC | 4 | | | |
| Type III-F | CCATATTGTGTACGATGCG | 5 | 0.04 | 280 | SCCmec III |
| Type III-R | CCTTAGTTGTCGTAACAGATCG | 6 | | | |
| Type IVa-F | GCCTTATTCGAAGAAACCG | 7 | 0.104 | 776 | SCCmec |
| Type IVa-R | CTACTCTTCTGAAAAGCGTCG | 8 | | | IVa |
| Type IVb-F | TCTGGAATTACTTCAGCTGC | 9 | 0.092 | 493 | SCCmec |
| Type IVb-R | AAACAATATTGCTCTCCCTC | 10 | | | IVb |
| Type IVc-F | ACAATATTTGTATTATCGGAGAGC | 11 | 0.078 | 200 | SCCmec |
| Type IVc-R | TTGGTATGAGGTATTGCTGG | 12 | | | IVc |
| Type IVd-F5 | CTCAAAATACGGACCCCAATACA | 13 | 0.28 | 881 | SCCmec |
| Type IVd-R6 | TGCTCCAGTAATTGCTAAAG | 14 | | | IVd |
| Type V-F | GAACATTGTTACTTAAATGAGCG | 15 | 0.06 | 325 | SCCmec V |
| Type V-R | TGAAAGTTGTACCCTTGACACC | 16 | | | |
| MecA147-F | GTG AAG ATA TAC CAA GTG ATT | 17 | 0.046 | 147 | mecA |
| MecA147-R | ATG CGC TAT AGA TTG AAA GGA T | 18 | | | |
| mecI-F | CCCTTTTTATACAATCTCGTT | 19 | 0.08 | 146 | Class A mec |
| mecI-R | ATATCATCTGCAGAATGGG | 20 | | | |
| IS1272-F | TATTTTTGGGTTTCACTCGG | 21 | 0.08 | 1305 | Class B mec |
| mecR1-R | CTCCACGTTAATTCCATTAATACC | 22 | | | |
| ^{a}ccrAB-β2 | ATTGCCTTGATAATAGCCITCT^{b} | 23 | 0.08 | | |
| ^{a}ccrAB-α2 | AACCTATATCATCAATCAGTACGT | 24 | 0.08 | 700 | Type 1 ccr |
| ^{a}ccrAB-α3 | TAAAGGCATCAATGCACAAACACT | 25 | 0.08 | 1000 | Type 2 ccr |
| ^{a}ccrAB-α4 | AGCTCAAAAGCAAGCAATAGAAT | 26 | 0.08 | 1600 | Type 3 ccr |
| ccrC-F | ATGAATTCAAAGAGCATGGC | 27 | 0.08 | 336 | Type 5 ccr |
| ccrC-R | GATTTAGAATTGTCGTGATTGC | 28 | | | |

^{a}The primer sequences adapted from Ito, 2001.
^{b}in the sequence refers to inosine and may be replaced by any one of A, G, T or C.

Detection or visualization of the PCR products after separation by gel electrophoresis may be accomplished by one of many available techniques known to those skilled in the art. In one embodiment, visualization may be accomplished using ethidium bromide staining and UV light. Other methods may include the use of labeled probes specific for the PCR products of interest.

As may be apparent to those skilled in the art, various modifications of the current invention are possible without departing from the scope of the invention, and are claimed within the scope of the present of the invention

### EXAMPLES

The examples below are carried out using standard techniques, which are well known and routine to those skilled in the art. These examples are intended to be illustrative, but not limiting, of the invention.

Simultaneous comparison of an assay of the present invention with the traditional PCR SCCmec typing method (including mec- and ccr-gene complex typing) and Oliveira's assay demonstrated 100% sensitivity and specificity when testing a large number of control strains. Further application of the assay in randomly selected local clinical isolates confirmed its feasibility and practicality.

### Example 1

### Bacterial Strains and Isolates

The SCCmec typing standard MRSA control strains, including type I (NCTC10442), type II (N315), type III (85/2082), type IVa (CA05), type IVb (8/6-3P), type IVc (MR108), type IVd (JCSC4469) and type V (WIS [WBG8318]-JCSC3624) (Table 1), were obtained from Dr. K. Hiramatsu and Dr. T. Ito at the Juntendo University in Tokyo, Japan (Ito, 2001 and 2004; Ma, 2002; Okuma, 2002). Additional SCCmec reference strains, including type I (COL and PER34) and type III (ANS46), were kindly provided by Dr. H. de Lencastre, the Rockefeller University, N.Y., USA (Oliveira, 2002). The Canadian epidemic MRSA reference strains, CMRSA-1 to 6, and strain N02-590 were provided by Dr. M. Mulvey, National Microbiology Laboratory, Health Canada, Winnipeg, Canada (Simor, 2002). Our local strains of MRSA belonging to various SCCmec types were obtained from Calgary Laboratory Services (CLS), Calgary, Alberta, Canada, and which had previously underwent phenotypic and genotypic analyses at the Centre for Antimicrobial Resistance, Calgary, Alberta, Canada (Table 3). Clinical MRSA isolates used for assessing the applicability and utility of our multiplex-PCR (M-PCR) assay were randomly selected from the CLS frozen clinical isolate stock collected over the August 1999 to November 2004 time period. Additional historical clinical MRSA strains were recovered from 5 tertiary acute-care teaching hospitals located in 4 cities in Canada (Winnipeg, Manitoba; Saskatoon, Saskatchewan; Calgary, Alberta; and Edmonton, Alberta) during the 1989-1994 period (Embil, 1994).

TABLE 3
Comparison of our assay with the traditional PCR and
a M-PCR SCCmec typing methods

| | Traditional PCR typing^{b} | | | | |
|---|---|---|---|---|---|
| | mec | ccr | | | |
| | complex | complex | SCCmec | Oliveira's | Our |
| Strain^{a} | type | type | type | M-PCR^{c} | Assay |
| NCTC10442 | B | 1 | I | I | I |
| COL | B | 1 | I | I | I |
| PER34 | B | 1 | I | I | I |
| N315 | A | 2 | II | II | II |
| CMRSA-2 | A | 2 | II | II | II |
| MRSA-80 | A | 2 | II | II | II |
| CLS-5153 | A | 2 | II | II | II |
| CLS-5371 | A | 2 | II | II | II |
| CLS-440 | A | 2 | II | II | II |
| CLS-72251 | A | 2 | II | II | II |
| CLS-69500 | A | 2 | II | II | II |
| CLS-68961 | A | 2 | II | II | II |
| CLS-6146 | A | 2 | II | II | II |
| CLS-4021 | A | 2 | II | II | II |
| CLS-2516 | A | 2 | II | II | II |
| CLS-52692 | A | 2 | II | II | II |
| CLS-19095 | A | 2 | II | II | II |
| 85/2082 | A | 3 | III | IIIB | III |
| ANS46 | A | 3 | III | III | III |
| CMRSA-3 | A | 3 | III | IIIA | III |
| CMRSA-6 | A | 3 | III | III | III |
| CLS-5861 | A | 3 | III | III | III |
| CLS-1777 | A | 3 | III | III | III |
| H163 | A | 3 | III | III | III |
| H478 | A | 3 | III | III | III |
| H527 | A | 3 | III | III | III |
| CA05 | B | 2 | IV | IV | IVa |
| N02-590 | B | 2 | IV | IV | IVa |
| CLS-2207 | B | 2 | IV | IV | IVa |
| CLS-3860 | B | 2 | IV | IV | IVa |
| CLS-2772 | B | 2 | IV | IV | IVa |
| CLS-1236 | B | 2 | IV | IV | IVa |
| CLS-884 | B | 2 | IV | IV | IVa |
| CLS-2772 | B | 2 | IV | IV | IVa |
| CLS-4550 | B | 2 | IV | IV | IVa |
| CLS-2245 | B | 2 | IV | IV | IVa |
| CLS-5897 | B | 2 | IV | IV | IVa |
| CLS-847 | B | 2 | IV | IV | IVa |
| CLS-846 | B | 2 | IV | IV | IVa |
| CLS-2525 | B | 2 | IV | IV | IVa |
| CLS-3497 | B | 2 | IV | IV | IVa |
| CLS-5401 | B | 2 | IV | IV | IVa |
| CLS-5381 | B | 2 | IV | IV | IVa |
| CLS-284 | B | 2 | IV | IV | IVa |
| 8/6-3P | B | 2 | IV | IV | IVb |
| CLS-4584 | B | 2 | IV | IV | IVb |
| CLS-5827 | B | 2 | IV | IV | IVb |
| CLS-6572 | B | 2 | IV | IV | IVb |
| MR108 | B | 2 | IV | IV | IVc |
| CLS-1040 | B | 2 | IV | IV | IVc |
| H434 | B | 2 | IV | IV | IVc |
| JCSC 4469 | B | 2 | IV | IV | IVd |
| CMRSA-5 | B | 2 | IV | IV | IVd |
| JCSC 3624 | C2 | 5 | V | III | V |
| WIS | | | | | |
| [WBG8318] | | | | | |

^{a}The SCCmec typing standard MRSA control strains: Type I (NCTC10442), Type II (N315), Type III (85/2082), Type IVa (CA05), Type Vb (8/6-3P), Type IVc (MR108), Type IVd (JCSC4469) and Type V (WIS [WBG8318]-JCSC3624); Additional SCCmec reference strains: type I (COL and PER34) and type III (ANS46); The Canadian epidemic MRSA reference strains: CMRSA-1 to 6, and strain N02-590; CLS- and H- are our local SCCmec type control strains.
^{b}Traditional PCR SCCmec typing methods (Ito, 2001 and 2004; Ma, 2002; Okuma, 2002).
^{c}Oliveira's multiplex PCR assay (Oliveira, 2002).

### Example 2

### Identification and Phenotypic Susceptibility Testing of Staphylococcal Isolates

Staphylococcal isolates were identified morphologically and biochemically by standard laboratory procedures (Murray, 2003). The coagulase plasma test (Remel, Lenexa, Kans., USA) was performed on organisms exhibiting typical staphylococcal colony morphology to allow for discrimination of S. aureus from coagulase-negative staphylococci (CoNS). Screening for methicillin and other antibiotic resistance phenotypes was done by VITEK (bioMerieux, Inc. Durham, N.C., USA) along with the NCCLS oxacillin agar screen, while confirmation of methicillin resistance was achieved using an in-house assay for the mecA gene (Hussain, 2000).

### Example 3

### Sequence Alignment and Primer Design

Gene targets, strains and accession numbers for each primer pair, as shown in Table 2 above, are as follows: type I—ORF E008 of strain NCTC10442 (AB033763), type II—kdpE of strain N315 (D86934), type III—ORF CZ049 of strain 85/2082 (AB37671), type IVa—ORF CQ002 of strain CA05 (AB063172), type IVb—ORF CM001 of strain 8/6-3P (AB063173), type IVc—ORF CR002 of strain MR108 (AB096217), type IVd—ORF CG001 of strain JCSC4469 (AB097677), type V—ORF V011 of strain JCSC3624 (AB12121), mecA—mecA gene of strain NCTC8325 (X52593), mecI—of strain N315, IS 1272 and mecR1-R—of strain CA05, ccrC—of strain JSCS 3624. The ccrAB primers are as previously described (Ito, 2001).

### Example 4

### DNA Extraction

Frozen bacteria were subcultured twice onto 5% sheep blood Columbia agar plates (PML Microbiologicals, Wilsonville, Oreg., USA) prior to DNA extraction. For rapid DNA extraction, 1-5 bacterial colonies were suspended in 50 μl of sterile distilled water and heated at 99° C. for 10 min. After centrifugation at 30,000× g for 1 min, 2 μl of the supernatant was used as template in a 25 μl PCR reaction (Zhang, 2004).

### Example 5

### PCR Amplification

The SCCmec M-PCR typing assay utilized 9 pairs of primers including the unique and specific primers for SCCmec types and subtypes I, II, III, IVa, IVb, IVc, IVd and V, and the primers for the mecA gene (Table 2).

The M-PCR assay used for characterization of mec-gene and ccr-gene complexes, respectively, contained 4 primers each (mecI-F, mecI-R, IS 1272-F and mecR1-R for mec-gene M-PCR, and ccrAB-p2, ccrAB-a2, ccrAB-a3 and ccrAB-a4 for ccr-gene M-PCR) (Table 2). The single target amplification PCR was used to determine type 5 ccr using ccrC-F and ccrC-R primers (Table 2). These primers and their respective concentrations used in the PCR are listed in Table 2.

All PCR assays were performed directly from bacterial suspensions obtained after the rapid DNA extraction method. An aliquot of 2 μl of this suspension was added to 23 μl of PCR mixture containing 50 mM KCl, 20 mM Tris-HCl (pH 8.4), 2.5 mM MgCl₂, 0.2 mM of each dNTP (DATP, dUTP, dGTP, dCTP) (Invitrogen Inc., Carlsbad, Calif., USA), variable concentrations of the respective primers (Table 2), and 1.0 unit of Platinum Taq DNA polymerase (Invitrogen Inc., Carlsbad, Calif., USA). The amplification was performed in a GeneAmp PCR system 9700 or 9600 Thermal Cycler (Applied Biosystems, Foster City, Calif., USA) beginning with an initial denaturation step at 94° C. for 5 min followed by 10 cycles of 94° C. for 45s (seconds), 65° C. for 45 s and 72° C. for 1.5 min and another 25 cycles of 94° C. for 45 s, 55° C. for 45 s and 72° C. for 1.5 min, ending with a final extension step at 72° C. for 10 min and followed by a hold at 4° C. For the single target amplification, PCR was run in 23 μl of PCR mixture but containing 0.2 μM of each primer, with cycling parameters beginning with an initial denaturation step at 94° C. for 5 min followed by 30 cycles of 94° C. for 1 min, 50° C. for 1 min and 72° C. for 2 min, ending with a final extension step at 72° C. for 10 min. For comparative purposes, SCCmec typing using Oliveira's method was performed using primer and PCR conditions described previously (Oliveira, 2002). All PCR assay runs incorporated a reagent control (without template DNA). The PCR amplicons were visualized using a UV light box after electrophoresis on a 2% agarose gel containing 0.5 μg/ml ethidium bromide.

### Example 6

### Limiting Dilution Experiments for Estimation of M-PCR Sensitivity

The sensitivity of amplification of various pairs of primers by M-PCR was estimated by limiting dilution experiments. Briefly, bacterial cultures from overnight growth at 37° C. on 5% sheep blood agar plates were suspended in sterile saline to a density corresponding to a 1.0 McFarland turbidity standard. These suspensions were then used to prepare serial 10-fold dilutions using sterile double distilled water. DNA extraction, using the rapid method described previously, was performed on 50 μl of each dilution. The standard M-PCR assay was performed to determine its sensitivity. The lower limits of detection (or minimal numbers of CFU detectable) of the target genes by M-PCR were then calculated based on correlation of the 1.0 McFarland standard to 3×10⁸ CFU/ml.

### Example 7

### Validation and Application of SCCmec Typing Method

The M-PCR assay was first optimized in the standard control strains and then validated with other control strains, and simultaneously compared with the traditional SCCmec typing methods including mec- and ccr-gene complex typing (methods above) and a previously described multiplex PCR assay, namely Oliveira's method (Oliveira, 2002). To assess the applicability and utility of the SCCmec typing assay, 453 randomly selected local clinical isolates from our MRSA clinical isolate frozen stock collection for the 1989-2004 time period were tested. To verify the assay's ability to differentiate MRSA from MSSA, comparison of our assay with standard phenotypic susceptibility testing (VITEK) and the conventional mecA gene PCR test (above methods) was conducted in 150 randomly selected local clinical MSSA isolates, in addition to the above 453 clinical MRSA isolates.

### Example 8

### Identification and Selection of Unique and Specific Loci and Primer Design for SCCmec Types and Subtypes

To design the SCCmec type- and subtype-unique and specific primers, an extensive BLAST sequence similarity search was conducted and was followed by comprehensive analyses and alignments of the S. aureus and MRSA genomes and SCCmec sequences currently available in the GenBank database. These loci consisted of open reading frames (ORFs) or sequence fragments, including ORF E008 of strain NCTC10442 (AB033763), kdpE of strain N315 (D86934), ORF CZ049 of strain 85/2082 (AB37671), ORF CQOO2 of strain CA05 (AB063172), ORF CM001 of strain 8/6-3P (AB063173), ORF CR002 of strain MR108 (AB096217), ORF CG001 of strain JCSC4469 (AB097677), and ORF V011 of strain JCSC3624 (AB12121), and were found to be unique and specific for SCCmec types and subtypes I, II, III, IVa, IVb, IVc, IVd and V, respectively. The corresponding SCCmec type- and subtype-unique and specific primers were designed (Table 2) and their uniqueness and specificity were further confirmed with a GenBank database BLAST search. Utilization of these primers in our novel M-PCR assay allowed us to specifically detect the currently described SCCmec types and subtypes of MRSA strains and clinical isolates.

### Example 9

### A New M-PCR for Typing and Subtyping SCCmec Types I-V, and Simultaneous Detection of Methicillin-Resistance (mecA Gene)

We developed a new and simple single M-PCR assay to determine (classify) SCCmec types and subtypes I, II, III, IVa, IVb, IVc, IVd and V, and simultaneously discriminate MRSA from MSSA. The M-PCR assay targeted the unique and specific loci of SCCmec types and subtypes I, II, III, IVa, IVb, IVc, IVd and V, with concomitant mecA gene detection, the latter serving as a determinant of methicillin resistance but also serving as an internal positive control for the assay. To ensure the individual primer pairs were adequate for the amplification of all nine loci (gene fragments), the single target PCR protocol with each individual primer pair was conducted prior to the M-PCR optimization, using 8 SCCmec standard control strains: type I (NCTC10442), type II (N315), type III (85/2082), type IVa (CA05), type IVb (8/6-3P), type IVc (MR108), type IVd (JCSC4469) and type V (WIS [WBG8318]-JCSC3624) (Table 1 and 3). Each individual PCR amplification reaction yielded the fragment of the expected size, i.e. 613, 398, 280, 776, 493, 200, 881, 325 and 147 bp for the unique and specific loci of SCCmec types and subtypes I, II, III, IVa, IVb, IVc, IVd and V, and mecA gene in their corresponding strains, respectively. The optimized M-PCR condition as described above was obtained through assaying different primer concentrations and other PCR reaction components. Amplification in a single M-PCR reaction produced distinct bands corresponding to their respective molecular sizes that were easily recognizable in agarose gels stained with ethidium bromide (FIG. 1).

### Example 10

### Sensitivity of M-PCR

The sensitivity of our M-PCR assay was examined in 8 SCCmec standard control strains {type I (NCTC10442), type II (N315), type III (85/2082), type IVa (CA05), type IVb (8/6-3P), type IVc (MR108), type IVd (JCSC4469) and type V (WIS [WBG8318]-JCSC3624)}. This assay was capable of detecting, with reproducibility, a band in ethidium bromide-stained gels at dilutions corresponding to 6×10⁴ CFU per PCR reaction for all 8 type- and subtype-specific genes. However, the sensitivity for the internal control mecA gene varied slightly depending on the strains examined, being 6×10⁵ CFU per PCR reaction for the strains NCTC 10442 (type I), JCSC4469 (type IVd) and WIS (type V), and 6×10⁴ CFU per PCR reaction for all other type or subtype strains [N315 (type ID, 85/2082 (type III), CA05 (type IVa), 8/6-3P (type IVb), MR108 (type IVc)]. This sensitivity is quite compatible with the single target PCR assay (1×10⁴˜6×10⁵) (data not shown), suggesting that the M-PCR assay is sufficiently robust.

### Example 11

### Validation of M-PCR Assay

To validate the M-PCR assay, we simultaneously compared our assay with the traditional PCR SCCmec typing scheme including mec- and ccr-gene complex typing and a previously described M-PCR assay (Oliveira, 2002). Validation of our assay was performed by testing a total of 54 well-characterized MRSA strains with known SCCmec types including type I (n=3), type II (n=14), type III (n=9), type IVa (n=18), type IVb (n=4), type IVc (n=3), type IVd (n=2), type V (n=1). We found a 100% concordance in typing SCCmec types I-IV between the PCR results of our M-PCR, traditional SCCmec typing method, and Oliveira's assay (results shown in Table 3) except for one type V strain. However, in the WIS strain (type V), both our assay and the traditional SCCmec typing method correctly identified this strain as SCCmec type V, but Oliveira's M-PCR falsely categorized the strain as SCCmec type III (Table 3). In addition, the M-PCR assay was able to further classify type IV strains into subtypes IVa, b, c, and d (Table 3).

To address our assay's ability in differentiating MRSA from MSSA, we tested 150 randomly selected local clinical MSSA isolates, in addition to the above 54 MRSA control strains and the 453 clinical MRSA isolates and found a mecA gene band (147 bp) in all MRSA isolates but not in any MSSA isolates, hence being 100% concordant with phenotypic susceptibility (VITEK) and conventional mecA gene PCR test results.

### Example 12

### Applicability and Accuracy of M-PCR

To assess the applicability and accuracy of the M-PCR assay, we further applied our SCCmec typing assay to test a total of 453 local clinical MRSA isolates randomly selected from our clinical stock collection for the 16-year period from 1989 to 2004. Among them, 235 (51.88%), 122 (26.93%), 74 (16.34%), 5 (1.1%) and 4 (0.88%) isolates belonged to SCCmec types and subtypes II, III, Va, IVb, and IVc, but no SCCmec types and subtypes I, IVd or V were found among the isolates tested. However, there were 13 (2.87%) isolates that were not-typeable using our assay, with 5 (1.10%) isolates having multiple bands and 8 (1.77%) isolates with amplification of only the mecA gene. These not-typeable isolates were further characterized using the traditional PCR SCCmec typing method and Oliveira's M-PCR assay. In 5 multiple-band isolates, one isolate presenting 2 bands of 200 bp and 280 bp (corresponding to types IVc and III by our new assay) was also not-typeable by the traditional PCR but was found to be type III by Oliveira's M-PCR, while the other 4 isolates with bands of 398 bp and either 613 bp or 200 bp (corresponding to types II and either type I or IVc by our new assay) were typed as types II in both other assays (Table 4), and may possibly represent un-described new subtypes of SCCmec type II. However, among the other 8 isolates with amplification of only the mecA gene, only one isolate (mecA-band 8) was determined to be type IV by both the traditional PCR SCCmec typing method and Oliveira's M-PCR assay, while the remaining (7 isolates) had incongruent typing results amongst the two other typing methods (Table 4), potentially representing new types or subtypes.

Both the traditional PCR SCCmec typing scheme and Oliveira's multiplex PCR technique are PCR methods targeting unique loci. Not-typeable MRSA isolates are encounted when using the traditional PCR SCCmec typing scheme and Oliveira's multiplex PCR technique but the nontypeability rate is variable. Ito et al used their traditional PCR typing method to type 617 MRSA isolates from Asian countries and found 5 (0.81%) strains were not-typeable (Ito et al., 2004). Perez-Roth et al (Perez-Roth, 2004) found 11 not-typeable clones out of 375 isolates (2.93%) (due to un-matching patterns) when typing MRSA clinical isolates during a 5-year period (1998-2002) in a Spanish hospital, and Chung et al (Chung, 2004) found 4 out of 113 isolates (3.54%) were not-typeable when typing MRSA strains recovered at a Florida hospital, when both groups of investigators used Oliveira's assay. The assay described herein was used to type 453 local clinical randomly selected isolates and found 13 (2.87%) not-typeable isolates. Except for one isolate (mecA-band 8), the remaining 12 isolates (Table 4) are potentially new types or subtypes. The explanation for these observations, as quoted by others, may be related to the presence of new structural types and subtypes or structural rearrangements and recombination of the mec element (Chung, 2004; Perez-Roth, 2004). Further investigations, including sequencing the mec element, are needed in order to characterize these not-typeable isolates.

TABLE 4
Comparison of SCCmec typing results for traditional PCR and Oliviera's
multiplex PCR assays for isolates not typeable by a multiplex PCR assay of the present
invention.

| | Assay^{b} | | | |
|---|---|---|---|---|
| | Specific PCR | | Traditional PCR | Oliveira's M- |
| Isolate^{a} | products (bp) | Corresponding to | typing^{c} | PCR^{d} |
| Multi-band 1 | 200 + 280 | Type IVc + III | Not-typeable | Type III |
| Multi-band 2 | 398 + 613 | Type II + I | Type II | Type II |
| Multi-band 3 | 398 + 613 | Type II + I | Type II | Type II |
| Multi-band 4 | 398 + 200 | Type II + IVc | Type II | Type II |
| Multi-band 5 | 398 + 200 | Type II + IVc | Type II | Type II |
| mecA-band 1 | 147 | mecA gene | Not-typeable | Type IV |
| mecA-band 2 | 147 | mecA gene | Not-typeable | Not-typeable |
| mecA-band 3 | 147 | mecA gene | Type IV | Type I |
| mecA-band 4 | 147 | mecA gene | Type IV | Not-typeable |
| mecA-band 5 | 147 | mecA gene | Type II | Type IV |
| mecA-band 6 | 147 | mecA gene | Type II | Type IV |
| mecA-band 7 | 147 | mecA gene | Type I | Not-typeable |
| mecA-band 8 | 147 | mecA gene | Type IV | Type IV |

^{a}Not-typeable isolates (multiple bands or single mecA gene band) using our new assay.
^{b}A multiplex PCR assay of the present invention.
^{c}Traditional PCR SCCmec typing methods (Ito, 2001 and 2004; Ma, 2002; Okuma, 2002).
^{d}Oliveira's multiplex PCR assay (Oliveira, 2002).

### References

The following references are referred in parenthesis in the above description and are incorporated herein as if reproduced in their entirety.
Ayliffe, G. A. (1997). The progressive intercontinental spread of methicillin-resistant Staphylococcus aureus. Clin Infect Dis, 24:S74-9.
Barber, M. (1961). Methicillin-resistant staphylococci. J Clin Pathol, 14:385-93.
Chung, M., G. Dickinson, H. De Lencastre, and A. Tomasz (2004). International clones of methicillin-resistant Staphylococcus aureus in two hospitals in Miami, Fla. J Clin Microbiol, 42:542-7.
Corkill, J. E., J. J. Anson, P. Griffiths, and C. A. Hart (2004). Detection of elements of the staphylococcal cassette chromosome (SCC) in a methicillin-susceptible (mecA gene negative) homologue of a fucidin-resistant MRSA. J Antimicrob Chemother, 54:229-31.
Crossley, K., D. Loesch, B. Landesman, K. Mead, M. Chem, and R. Strate (1979). An outbreak of infections caused by strains of Staphylococcus aureus resistant to methicillin and aminoglycosides. I. Clinical studies. J Infect Dis, 139:273-9.
Embil, J., K. Ramotar, L. Romance, M. Alfa, J. Conly, S. Cronk, G. Taylor, B. Sutherland, T. Louie, E. Henderson, et al. (1994). Methicillin-resistant Staphylococcus aureus in tertiary care institutions on the Canadian prairies 1990-1992. Infect Control Hosp Epidemiol, 15:646-51.
Enright, M. C., D. A. Robinson, G. Randle, E. J. Feil, H. Grundmann, and B. G. Spratt (2002). The evolutionary history of methicillin-resistant Staphylococcus aureus (MRSA). Proc Natl Acad Sci USA, 99:7687-92.
Fluit, A. C., J. Verhoef, and F. J. Schmitz (2001). Frequency of isolation and antimicrobial resistance of gram-negative and gram-positive bacteria from patients in intensive care units of 25 European university hospitals participating in the European arm of the SENTRY Antimicrobial Surveillance Program 1997-1998. Eur J Clin Microbiol Infect Dis, 20:617-25.
Hussain, Z., L. Stoakes, V. Massey, D. Diagre, V. Fitzgerald, S. El Sayed, and R. Lannigan (2000). Correlation of oxacillin MIC with mecA gene carriage in coagulase-negative staphylococci. J Clin Microbiol, 38:752-4.
Ito, T., Y. Katayama, and K. Hiramatsu (1999). Cloning and nucleotide sequence determination of the entire mec DNA of pre-methicillin-resistant Staphylococcus aureus N315. Antimicrob Agents Chemother, 43:1449-58.
Ito, T., Y. Katayama, K. Asada, N. Mori, K. Tsutsumimoto, C. Tiensasitom, and K. Hiramatsu (2001). Structural comparison of three types of staphylococcal cassette chromosome mec integrated in the chromosome in methicillin-resistant Staphylococcus aureus. Antimicrob Agents Chemother, 45:1323-36.
Ito, T., K. Okuma, X. X. Ma, H. Yuzawa, and K. Hiramatsu (2003). Insights on antibiotic resistance of Staphylococcus aureus from its whole genome: genomic island SCC. Drug Resist Updat, 6:41-52.
Ito, T., X. X. Ma, F. Takeuchi, K. Okuma, H. Yuzawa, and K. Hiramatsu (2004). Novel type V staphylococcal cassette chromosome mec driven by a novel cassette chromosome recombinase, ccrC. Antimicrob Agents Chemother, 48:2637-51.
Ito, T., X. Ma, Y. Kondo, P. Changtrakool, S. Traklsomboon, C. Tiensasitom, M. Jamklang, T. Chavalit, J. Song, and K. Hiramatsu, Abstr. 44^{th} Intersci. Conf. Antimicrob. Agents Chemother., abstr. and poster 115, 2004.
Jevons, M. P. (1961). “Celbenin”-resistant staphylococci. British Medical Journal, 1:124-125.
Katayama, Y., F. Takeuchi, T. Ito, X. X. Ma, Y. Ui-Mizutani, I. Kobayashi, and K. Hiramatsu (2003). Identification in methicillin-susceptible Staphylococcus hominis of an active primordial mobile genetic element for the staphylococcal cassette chromosome mec of methicillin-resistant Staphylococcus aureus. J Bacteriol, 185:2711-22.
Lindsay, J. A., and M. T. Holden (2004). Staphylococcus aureus: superbug, super genome? Trends Microbiol, 12:378-85.
Livermore, D. M. (2000). Antibiotic resistance in staphylococci. Int J Antimicrob Agents, 16, Suppl 1:S3-10.
Luong, T. T., S. Ouyang, K. Bush, and C. Y. Lee (2002). Type 1 capsule genes of Staphylococcus aureus are carried in a staphylococcal cassette chromosome genetic element. J Bacteriol, 184:3623-9.
Ma, X. X., T. Ito, C. Tiensasitorn, M. Jamklang, P. Chongtrakool, S. Boyle-Vavra, R. S. Daum, and K. Hiramatsu (2002). Novel type of staphylococcal cassette chromosome mec identified in community-acquired methicillin-resistant Staphylococcus aureus strains. Antimicrob Agents Chemother, 46:1147-52.
Mongkolrattanothai, K., S. Boyle, T. V. Murphy, and R. S. Daum (2004). Novel non-mecA-containing staphylococcal chromosomal cassette composite island containing pbp4 and tagF genes in a commensal staphylococcal species: a possible reservoir for antibiotic resistance islands in Staphylococcus aureus. Antimicrob Agents Chemother, 48:1823-36.
Murray, P. R. (2003). Manual of Clinical Microbiology, 8th ed. American Society for Microbiology Press, Washington, D.C., USA.
O'Brien, F. G., T. T. Lim, F. N. Chong, G. W. Coombs, M. C. Enright, D. A. Robinson, A. Monk, B. Said-Salim, B. N. Kreiswirth, and W. B. Grubb (2004). Diversity among community isolates of methicillin-resistant Staphylococcus aureus in Australia. J Clin Microbiol, 42:3185-90.
Okuma, K., K. Iwakawa, J. D. Tumidge, W. B. Grubb, J. M. Bell, F. G. O'Brien, G. W. Coombs, J. W. Pearman, F. C. Tenover, M. Kapi, C. Tiensasitom, T. Ito, and K. Hiramatsu (2002). Dissemination of new methicillin-resistant Staphylococcus aureus clones in the community. J Clin Microbiol, 40:4289-94.
Oliveira, D. C., A. Tomasz, and H. de Lencastre (2001). The evolution of pandemic clones of methicillin-resistant Staphylococcus aureus: identification of two ancestral genetic backgrounds and the associated mec elements. Microb Drug Resist, 7:349-61.
Oliveira, D. C., and H. de Lencastre (2002). Multiplex PCR strategy for rapid identification of structural types and variants of the mec element in methicillin-resistant Staphylococcus aureus. Antimicrob Agents Chemother, 46:2155-61.
Panlilio, A. L., D. H. Culver, R. P. Gaynes, S. Banerjee, T. S. Henderson, J. S. Tolson, and W. J. Martone (1992). Methicillin-resistant Staphylococcus aureus in U.S. hospitals, 1975-1991. Infect Control Hosp Epidemiol, 13:582-6.
Perez-Roth, E., F. Lorenzo-Diaz, N. Batista, A. Moreno, and S. Mendez-Alvarez (2004). Tracking methicillin-resistant Staphylococcus aureus clones during a 5-year period (1998 to 2002) in a Spanish hospital. J Clin Microbiol, 42:4649-56.
Robinson, D. A., and M. C. Enright (2003). Evolutionary models of the emergence of methicillin-resistant Staphylococcus aureus. Antimicrob Agents Chemother, 47:3926-34.
Robinson, D. A., and M. C. Enright (2004). Multilocus sequence typing and the evolution of methicillin-resistant Staphylococcus aureus. Clin Microbiol Infect, 10:92-7.
Simor, A. E., M. Offier-Agostini, E. Bryce, A. McGeer, S. Paton, and M. R. Mulvey (2002). Laboratory characterization of methicillin-resistant Staphylococcus aureus in Canadian hospitals: results of 5 years of National Surveillance, 1995-1999. J InfectDis, 186:652-60.
Vandenesch, F., T. Naimi, M. C. Enright, G. Lina, G. R. Nimmo, H. Heffeman, N. Liassine, M. Bes, T. Greenland, M. E. Reverdy, and J. Etienne (2003). Community-acquired methicillin-resistant Staphylococcus aureus carrying Panton-Valentine leukocidin genes: worldwide emergence. Emerg Infect Dis, 9:978-84.
Voss, A., D. Milatovic, C. Wallrauch-Schwarz, V. T. Rosdahl, and I. Braveny (1994). Methicillin-resistant Staphylococcus aureus in Europe. Eur J Clin Microbiol Infect Dis, 13:50-5.
Zhang, K., J. Sparling, B. L. Chow, S. Elsayed, Z. Hussain, D. L. Church, D. B. Gregson, T. Louie, and J. M. Conly (2004). New quadriplex PCR assay for detection of methicillin and mupirocin resistance and simultaneous discrimination of Staphylococcus aureus from coagulase-negative staphylococci. J Clin Microbiol, 42:4947-55.

What is claimed is:

1. A multiplex PCR SCCmec typing assay for Staphylococcus aureus SCCmec types I, II, III, subtypes IVa, IVb, IVc, IVd and V, and MRSA and MSSA, comprising the steps of:
(a) obtaining an isolate of a sample of S. aureus;
(b) amplifying a loci unique to each type and subtype by a PCR technique using amplification primers selective for each of said loci; and
(c) detecting the PCR amplicons, and determining the type and subtype of the S. aureus isolate.

2. The assay of claim 1, wherein the amplification primers comprises one or both of each of the following primer pairs: SEQ ID NO 1 and 2, SEQ ID NO 3 and 4, SEQ ID NO 5 and 6, SEQ ID NO 7 and 8, SEQ ID NO 9 and 10, SEQ ID NO 11 and 12, SEQ ID NO 13 and 14, SEQ ID NO 15 and 16, and SEQ ID NO 17 and 18.

3. The assay of claim 1 or 2 further comprising amplification primers selective for the mec gene complex and the ccr gene complex.

4. The assay of claim 3 wherein the mec gene complex amplification primers comprises one or more of SEQ ID NO 19-22 and the ccr gene complex amplification primers comprises one or more of SEQ ID NO 23-28.

5. The assay of claim 1 wherein the loci unique to each type and subtype comprises: ORF E008 of strain NCTC10442 (AB033763), kdpE of strain N315 (D86934), ORF CZ049 of strain 85/2082 (AB37671), ORF CQ002 of strain CA05 (AB063172), ORF CM001 of strain 8/6-3P (AB063173), ORF CR002 of strain MR108 (AB096217), ORF CG001 of strain JCSC4469 (AB097677), ORF V011 of strain JCSC3624 (AB12121), mecA—mecA gene of strain NCTC8325 (X52593), mecI—of strain N3 15, IS 1272 and mecR1-R—of strain CA05, ccrC—of strain JSCS 3624.

6. A multiplex PCR SCCmec typing assay kit for Staphylococcus aureus SCCmec types I, II, III, subtypes IVa, IVb, IVc, IVd, and V, and MRSA and MSSA, said kit comprising one or both of each of the following primer pairs: SEQ ID NO 1 and 2, SEQ ID NO 3 and 4, SEQ ID NO 5 and 6, SEQ ID NO 7 and 8, SEQ ID NO 9 and 10, SEQ ID NO 11 and 12, SEQ ID NO 13 and 14, SEQ ID NO 15 and 16, and SEQ ID NO 17 and 18.

7. The kit of claim 6 further comprising amplification primers selective for the mec gene complex and the ccr gene complex.

8. The kit of claim 7 wherein the mec gene complex amplification primers comprises one or more of SEQ ID NO 19-22 and the ccr gene complex amplification primers comprises one or more of SEQ ID NO 23-28.


SEQUENCE LISTING (28 sequences)

SEQ ID NO: 1 - DNA, 23 bp, Staphylococcus aureus
gctttaaagagtgtcgttacagg

SEQ ID NO: 2 - DNA, 22 bp, Staphylococcus aureus
gttctctcatagtatgacgtcc

SEQ ID NO: 3 - DNA, 19 bp, Staphylococcus aureus
cgttgaagatgatgaagcg

SEQ ID NO: 4 - DNA, 22 bp, Staphylococcus aureus
cgaaatcaatggttaatggacc

SEQ ID NO: 5 - DNA, 19 bp, Staphylococcus aureus
ccatattgtgtacgatgcg

SEQ ID NO: 6 - DNA, 22 bp, Staphylococcus aureus
ccttagttgtcgtaacagatcg

SEQ ID NO: 7 - DNA, 19 bp, Staphylococcus aureus
gccttattcgaagaaaccg

SEQ ID NO: 8 - DNA, 21 bp, Staphylococcus aureus
ctactcttctgaaaagcgtcg

SEQ ID NO: 9 - DNA, 20 bp, Staphylococcus aureus
tctggaattacttcagctgc

SEQ ID NO: 10 - DNA, 20 bp, Staphylococcus aureus
aaacaatattgctctccctc

SEQ ID NO: 11 - DNA, 24 bp, Staphylococcus aureus
acaatatttgtattatcggagagc

SEQ ID NO: 12 - DNA, 20 bp, Staphylococcus aureus
ttggtatgaggtattgctgg

SEQ ID NO: 13 - DNA, 23 bp, Staphylococcus aureus
ctcaaaatacggaccccaataca

SEQ ID NO: 14 - DNA, 20 bp, Staphylococcus aureus
tgctccagtaattgctaaag

SEQ ID NO: 15 - DNA, 23 bp, Staphylococcus aureus
gaacattgttacttaaatgagcg

SEQ ID NO: 16 - DNA, 22 bp, Staphylococcus aureus
tgaaagttgtacccttgacacc

SEQ ID NO: 17 - DNA, 21 bp, Staphylococcus aureus
gtgaagatataccaagtgatt

SEQ ID NO: 18 - DNA, 22 bp, Staphylococcus aureus
atgcgctatagattgaaaggat

SEQ ID NO: 19 - DNA, 21 bp, Staphylococcus aureus
ccctttttatacaatctcgtt

SEQ ID NO: 20 - DNA, 19 bp, Staphylococcus aureus
atatcatctgcagaatggg

SEQ ID NO: 21 - DNA, 20 bp, Staphylococcus aureus
tatttttgggtttcactcgg

SEQ ID NO: 22 - DNA, 24 bp, Staphylococcus aureus
ctccacgttaattccattaatacc

SEQ ID NO: 23 - DNA, 22 bp, Staphylococcus aureus
  misc_feature (19)..(19): n is a, c, g, or t
attgccttgataatagccntct

SEQ ID NO: 24 - DNA, 24 bp, Staphylococcus aureus
aacctatatcatcaatcagtacgt

SEQ ID NO: 25 - DNA, 24 bp, Staphylococcus aureus
taaaggcatcaatgcacaaacact

SEQ ID NO: 26 - DNA, 23 bp, Staphylococcus aureus
agctcaaaagcaagcaatagaat

SEQ ID NO: 27 - DNA, 20 bp, Staphylococcus aureus
atgaattcaaagagcatggc

SEQ ID NO: 28 - DNA, 22 bp, Staphylococcus aureus
gatttagaattgtcgtgattgc